(19) United States
(12) Patent Application Publication
(10) Pub. No.: US 2008/0064058 A1
(43) Pub. Date: Mar. 13, 2008

(54) Method and System for Detection of Trichinella Larvae in Meat Samples

(75) Inventors: Christian Moliin Outzen Moulin Outzen Kapel, Rungsted Kyst (DK); Nils Hammeken, Olstykke (DK); Jens Michael Carstensen, Bjaeverskov (DK); Leif Dalum, Rungsted Kyst (DK)

Correspondence Address: SEED INTELLECTUAL PROPERTY LAW GROUP PLLC, 701 FIFTH AVE, SUITE 5400, SEATTLE, WA, 98104, US

(73) Assignee: ParaTest ApS, Horsholm (DK)

(21) Appl. No.: 11/664,359

(22) PCT Filed: Sep. 29, 2005
(86) PCT No.: PCT/DK05/00619

§ 371 Date: Nov. 6, 2007

(30) Foreign Application Priority Data
Sep. 30, 2004 (DK) PA 2004 01492

(51) Int. Cl. C12Q 1/04

(52) U.S. Cl. 435/34

(57) ABSTRACT

The present invention relates to a method and a system for detection of Trichinella larvae in meat samples. The method comprises digestion of an amount of meat to be tested for Trichinella larvae, where the digestion process results in a digested fluid, filtering the digested fluid by use of a first filter being designed to retain Trichinella larvae, and analysing by use of optical inspection the area of the first filter being used for the filtration of the digested fluid, where the analysis is directed against identification of Trichinella larvae. It is preferred that the optical inspection comprises recording one or more images of the filtration area of the first filter being used for the filtration process. It is also preferred that a plurality of images are recorded, where the plurality of recorded images represents a plurality of parts of the filtration area of the first filter, and the recorded images may represent the whole area being used for the filtration process. A digital camera may used for obtaining the images on a digital form. The camera and a filtration plane being defined by the filtration area of the first filter may be arranged movably relative to each other. It is preferred that the filtration area or a part of the filtration area is represented by a multitude of recorded images. Here the multitude of recorded images may represent a set of different illuminations of the filtration area of the first filter. The analysing process may also comprise an image analysis of the recorded images, and the image analysis may include an analysis of surface reflectance of objects being recorded at different wavelengths. The image analysis may also include a shape and size analysis of recorded objects.

### FIELD OF THE INVENTION

The present invention relates to a method and a system for detection of Trichinella larvae in meat samples, and particularly the invention relates to a method and a system comprising a digestion of meat samples, where detection of Trichinella larvae is performed by optical inspection.

### BACKGROUND OF THE INVENTION

The prior art detection technique is constituted by the so-called “digestion” method. This method is used extensively for the testing for the presence of Trichinella larvae in muscle tissue of pigs. This technique involves a mechanically assisted enzymatic digestion of tissue samples (usually about 100×1 g or 20×5 g) in an acidic pepsin solution, followed by sedimentation or sieving and straining the resulting solution, then viewing the resultant strained matter under a microscope.

Guidelines for Trichinella control have been published by EU (European Community, 1984), USA (Office of the Federal Register, 1990), Russia (Tret'Yakov, 1972), and OIE (Office Internationale des Epizooties, 1999). The International Commission on Trichinellosis (ICT) have condensed all available guidelines into a set of recommendations on methods for the control of Trichinella in domestic and wild animals (Gamble et al., 2000, http://www.med.unipi.it/ict/Recomm.htm). In this publication, ICT recommends a digestion technique that combines blending or grinding to disrupt the tissue followed by digestion at 45° C. using a magnetic stirrer, which is comparable to method 6 of the present EU legislation, and the “gold standard” in the new legislation to come into force early 2006 with the new hygiene directive.

For any digestion procedure used for meat inspection for Trichinella, the following critical points are stated in the ICT recommendations:

1. A verifiable system of sample collection and identification must be maintained. The process must assure that samples of 1 g or greater size originate from the appropriate number of pigs and that samples are clearly identified back to individual pigs.

2. Digestion fluid must be consistent in quality and prepared in a manner that do not reduce the activity of the pepsin. The most critical step in the preparation is the addition of the hydrochloric acid to the water prior to addition of pepsin to prevent degradation of the enzyme.

3. The temperature maintained during the digestion process should not exceed 45° C.

4. Following digestion, no undigested muscle tissue should remain. Digestion must be completed to ensure the integrity of the test.

5. Sedimentation procedures and times should be conducted to maximize the recovery of larvae. Existing methods employ sedimentation time of 30 min. for complete sedimentation of larvae.

6. Digested samples must be clarified sufficiently to allow visualization of larvae. Newsprint should be readable though a Petri dish with the sample.

7. Microscopic examination should be conducted at 15-40× magnification.

8. Digest should be examined prior to release of the carcass.

9. Records are kept that ensures the accurate identification of sample and carcass.

The pooled digestion techniques, as described in the EU legislation, are considered to be capable of detecting carcasses harbouring the minimum of larval load that would cause clinical illness in people. In the EU legislation, two methods involving a filtration of the digested mixture are described: Method 5 “Stomacher with filtration” and method 7 “Trichomatic 35”, where the “Trichomatic 35” comprises an automated digestion unit, which however can only digest up to 35 g of tissue at the same time. For both of these methods, the membrane which has been used for the filtration of the digested mixture has to be manually inspected under microscope.

Thus, all of the above-mentioned tests have a need for a person to perform inspection by use of a microscope. Such tests have proven to require continuous concentration of the trained inspector for the detection of larvae. Likewise, many inspectors have only seen a Trichinella larva once in their life during their initial training. The likelihood of finding Trichinella larvae in pigs send to a slaughterhouse is indeed very low (99.999% of EU's pig production is with negligible risk of Trichinella). Today, there are no legislative requirements or practice for repeated training of inspectors or for the use of proficiency panels to validate the existing procedures.

All present methods approved by EU and other legislative agencies are very labour intensive and require several mechanical steps and manual addition of chemical, and there is no requirement for quality control on the actual procedures.

Thus, there is a need for a test, where the routine optical inspection of the isolated matter may be performed without microscopy by inspector, and which allows automatic sampling and manipulation of samples.

### SUMMARY OF THE INVENTION

According to the present invention, there is provided a method for detection of Trichinella larvae in meat samples comprising:

a) digesting an amount of meat to be tested for parasite larvae, said digestion process resulting in a digested fluid,

b) filtering the digested fluid by use of a first filter being designed to retain Trichinella larvae, and

c) analysing by use of optical inspection the area of the first filter being used for the filtration of the digested fluid, said analysing being directed against identification of Trichinella larvae.

It is preferred that the optical inspection comprises recording one or more images of the filtration area of the first filter being used for the filtration process. Here, a plurality of images may be recorded thereby representing a plurality of parts of the filtration area of the first filter. It is preferred that the recorded image or the plurality of recorded images represent the whole area being used for the filtration process

Thus, by using optical inspection of the filter used for retaining the parasite larvae, it is possible to use an automated analysis based on cumulative recorded images covering the entire filter.

It is preferred that a camera is used for recording said one or more images, and the camera may be a digital camera whereby the recorded one or more images may be given on a digital form. It is preferred that the camera and a filtration plane being defined by the filtration area of the first filter are arranged movably relative to each other, whereby the plurality of images of the parts of the filtration area can be obtained. Here, the camera may be arranged movably in a plane substantially parallel to a plane being defined by the filtration area of the first filter.

It is within an embodiment of the invention that the filtration area or a part of the filtration area is represented by a multitude of recorded images. When a plurality of images are recorded thereby representing a plurality of parts of the filtration area of the first filter, it is within a preferred embodiment that each of the plurality of parts of the filtration area being represented by a recorded image is represented by a multitude of recorded images.

It is also within an embodiment of the invention that a multitude of recorded images representing the filtration area or a part of the filtration area represents a set of different illuminations of the filtration area of the first filter. It is preferred that each of the multitude of recorded images representing the filtration area or a part of the filtration area represents the same set of different illuminations of the filtration area of the first filter. Preferably, the set of illuminations used for the filtration area may comprise one or more illuminations selected from the list including: dark field illumination, bright field illumination, diffuse illumination and backlight illumination.

It is also within an embodiment of the invention that the set of illuminations used for the filtration area comprises illumination or diffuse illumination at one or more different wavelengths. Here, the illumination(s) or diffuse illumination(s) may be selected from a list of wavelengths representing the visible, the ultraviolet and the infrared part of the electromagnetic spectrum.

According to an embodiment of the present invention, the analysing process may comprise an image analysis of the recorded image(s). Here, the image analysis may be performed by use of a computer, calculator or calculating means being adapted to perform an analysis on the basis of the recorded image(s). The image analysis may include a shape and size analysis of recorded objects. Here the recorded objects may not be part of the first filter. It is also within an embodiment of the invention that the image analysis may include an analysis of surface reflectance of objects being recorded at different wavelengths. It is furthermore within an embodiment of the invention that the image analysis may include an analysis of surface fluorescence of objects being recorded.

According to an embodiment of the invention, the image analysis may comprise a calibration process of the computer, calculator or calculator means being used or adapted to perform the image analysis. The calibration process may include recording of images of the first filter or parts of the first filter, where the first filter or parts of the first filter have not been used for any filtration. The calibration may be based on the filter mesh size or the distance between the threads in the net filter or sieve.

In order to obtain an efficient digestion process, it is preferred that the amount of meat to be digested is divided into smaller pieces before the digestion process. The dividing of the meat into smaller pieces may comprise pressing the amount of meat through holes or openings of a predetermined area. Here, the holes or openings may have a maximum inner diameter in the range of 2-5 mm, such as around 3 mm.

It is also within an embodiment of the invention that the amount of meat to be digested is minced in a blender before the digestion process.

In order to obtain an efficient digestion process, it is also within an embodiment of the invention to use a magnetic stirrer to stir the digestion solution during the digestion process.

When filtering the digested fluid, it is preferred the digested fluid is filtered by use of a second filter before being filtered by use of the first filter. Here, the second filter should have a larger mesh size than the first filter, whereby the parasite larvae to be identified can pass through the second filter. The second filter may preferably have a mesh size in the range of 160-300 μm, such as in the range of 160-200 μm, such as around 180 μm.

The first filter may have a mesh size in the range of 20-60 μm or in the range of 25-50 μm. Preferably, the first filter may have a mesh size in the range of 30-40 μm, such as around 35 μm.

It should be understood that the method of detection according to the present invention may be used for different sorts of meats. Thus, the amount of meat to be digested may originate from pig, horse or game.

The method of the present invention may also include an embodiment comprising an automated sampling of specific amounts of tissue from specified muscle tissue of degutted slaughter pigs thereby providing the amount of meat to be digested. Here, an accurate amount of specified muscle tissue may be sampled by a robot arm from the body of the degutted slaughter pig by the use of an “ice-scoop” formed forceps.

According to an embodiment of the invention, the digestion process may be automatically controlled.

According to an embodiment of the invention, the first filter may be arranged on a roll, and the used filtration area of the first filter may be moved from an area of a filtration arrangement to a camera area thereby leaving an unused filtration area of the first filter at the area of the filtration arrangement. When using a second filter, it is preferred that the second filter is arranged on a roll, and that a used filtration area of the second filter is moved from the area of the filtration arrangement after the filtration process to thereby leave an unused filtration area of the second filter at the area of the filtration arrangement, said second filter being arranged above the first filter at the filtration arrangement.

According to the present invention there is also provide a system for detection of Trichinella larvae in meat, said system comprising:

a digestion container for digesting an amount of meat to be tested for Trichinella larvae, said digestion process resulting in a digested fluid,

a first filter for filtering the digested fluid, said first filter being designed to retain Trichinella larvae, and

an analyser or analysing means for analysing by use of optical inspection the area of the first filter being used for the filtration of the digested fluid, said analysing being directed against identification of Trichinella larvae.

It is preferred that the analyser or analysing means comprises an image recorder for recording one or more images of the filtration area of the first filter being used for said filtration process. Here, the image recorder may be adapted for recording a plurality of images, said plurality of recorded images representing a plurality of parts of the filtration area of the first filter. It is preferred that the image recorder is adapted for recording one or more images representing the whole filtration area of the first filter.

The image recorder may comprise a camera for recording said one or more images, and also here the camera may be a digital camera and the recorded one or more images may be given on a digital form. Also, for the system of the invention it is preferred that the camera and a filtration plane being defined by the filtration area of the first filter are arranged movably relative to each other, whereby the plurality of images of the parts of the filtration area can be obtained. Also here, the camera may be arranged movably in a plane substantially parallel to a plane being defined by the filtration area of the first filter.

It is within an embodiment of the system of the invention that the image recorder is adapted to record a multitude of images of the filtration area or a part of the filtration area. When the image recorder is adapted for recording a plurality of images, with said plurality of recorded images representing a plurality of parts of the filtration area of the first filter, it is preferred that the image recorder is adapted to record a multitude of images of each of the plurality of parts of the filtration area being represented by a recorded image.

The system of the invention may comprise one or more illumination devices for providing different illuminations of the filtration area of the first filter. Here, a multitude of recorded images representing the filtration area or a part of the filtration area may represent a set of different illuminations of the filtration area of the first filter. Also for the system of the invention, the set of illuminations used for the filtration area may comprise one or more illuminations selected from the list including: dark field illumination, bright field illumination, diffuse illumination and backlight illumination.

It is preferred that the one or more illumination devices for providing different illuminations of the filtration area is adapted for providing illumination or diffuse illumination at one or more different wavelengths. Here, the illumination(s) or diffuse illumination(s) may be selected from a list of wavelengths representing the visible, the ultraviolet and the infrared part of the electromagnetic spectrum.

According to an embodiment of the system of the invention, the analyser or analysing means may be adapted for performing an image analysis of the recorded image(s), and the analyser or analysing means may comprise a computer, calculator or calculating means being adapted to perform an analysis on the basis of the recorded image(s). The analyser or analysing means may be adapted for performing a shape and size analysis of recorded objects, which recorded objects need not being part of the first filter. It is also within an embodiment of the system of the invention that the analyser or analysing means may be adapted for performing an analysis of surface reflectance of objects being recorded at different wavelengths. It is furthermore within an embodiment of the system of the invention that the analyser or analysing means may be adapted for performing an analysis of surface fluorescence of objects being recorded.

According to an embodiment of the system of the invention, the analyser or analysing means may be adapted for performing a calibration process of the computer or calculating means being used or adapted to perform the image analysis. The analyser or analysing means may be adapted to perform the calibration process based on recording of images of the first filter or parts of the first filter, where the first filter or parts of the first filter have not been used for any filtration. The calibration process may further be based on the filter mesh size or the distance between the threads in the net filter or sieve.

The system of the present invention also covers an embodiment, wherein the system further comprises a grinding compartment having a divider for dividing the amount of meat to be digested into smaller pieces before the digestion process. The divider may be adapted for pressing the amount of meat through holes or openings of a predetermined area. Also here, the holes or openings may have a maximum inner diameter in the range of 2-5 mm, such as around or not larger than 3 mm.

The system of the present invention also covers an embodiment, wherein the system as an alternative or supplement to the grinding compartment comprises a blending compartment having a blender for mincing the meat or part or the meat to be digested.

According to a preferred embodiment, the system of the invention further comprises a second filter for straining or filtering the digested mixture before being filtered by use of the first filter, with the second filter having a larger mesh size than the first filter, whereby the parasite larvae to be identified can pass through the second filter. Also here, the second filter may have a mesh size in the range of 160-300 μm, such as in the range of 160-200 μm, such as about 180 μm, and also here the first filter may have a mesh size in the range of 20-60 μm or in the range of 25-50 μm. Preferably, the first filter may have a mesh size in the range of 30-40 μm, such as about 35 μm.

Also for the system of the invention, it is within a preferred embodiment that system comprises a magnetic stirrer for stirring the digestion solution during the digestion process, and it is also within an embodiment that the system comprises a controller for automatically controlling the digestion process.

For the system of the invention, it is also within a preferred embodiment that the first filter is arranged on a roll so that the used filtration area of the first filter can be moved from an area of a filtration arrangement to a camera area thereby leaving an unused filtration area of the first filter at the area of the filtration arrangement. When the system comprises a second filter, the second filter may be arranged on a roll so that a used filtration area of the second filter can be moved from the area of the filtration arrangement after the filtration process to thereby leave an unused filtration area of the second filter at the area of the filtration arrangement, said second filter being arranged above the first filter at the filtration arrangement.

From the above discussion, it should be understood that the present invention relates to a method and a system for detection of parasite larvae, which may be used for automated meat sampling, isolation and detection of Trichinella larvae in meat samples. The invention also relates to a method and a system for detection of Trichinella larvae, which may comprise a site-specific recovery of muscle samples, and an enzymatic mechanically assisted digestion of pools of meat samples, where the optical detection of parasite larvae is performed by computerised analysis of recorded images.

The invention is described in more detail in the following with reference to the accompanying drawings.

### BRIEF DESCRIPTION OF THE DRAWINGS

FIG. 1 is a block diagram illustrating a Trichinella larvae detection system according to an embodiment of the invention,

FIG. 2 shows a schematic view of a meat-grinding device according to an embodiment of the invention,

FIG. 3 shows a schematic view of an embodiment of a digestion container to be used in the system of FIG. 1,

FIG. 4 shows a schematic view of an arrangement of filters, filtration arrangement and camera according to an embodiment of the invention,

FIG. 5 shows a schematic view of an embodiment of a filtration arrangement according to an embodiment of the invention,

FIG. 6 shows a schematic view of a camera and illumination arrangement for optical inspection of a filter according to an embodiment of the invention,

FIG. 7 is a flow chart showing steps of an identification process according to an embodiment of the invention, and

FIG. 8 is a flow chart illustrating tracing of sampled carcasses, which have been detected as been infected by use of a detection system according to an embodiment of the invention.

### DETAILED DESCRIPTION OF THE INVENTION

The detection method and system according to the present invention may allow for the automated digestion of meat and computerized identification of Trichinella larvae. In the following, a detailed description is given for a preferred embodiment of the invention. In the design and function of the detection system of the invention, the critical points for digestion procedures as stated above in the ICT recommendations have been considered carefully.

Compartmentalization

The detection system of the invention may consist of several compartments to allow the customisation to any given legislation requirements in a particular country. The fully equipped detection system is intended to fulfil the requirements of any large-scale industrialized pig meat industry, typically in EU or North America. For high capacity testing, the detection system may be designed in a way that several digestion compartments can be connected to the same filtration and identification compartment.

According to an embodiment of the invention, the detection system may include the following four compartments:

1. The Sampling Compartment

This compartment is based on the automated removal of internal organs already being implemented on a few Danish slaughterhouses. After scanning of the pig (defining the size of the pig) and removal of the internal organs, an arm with a cutting device will cut a 1 g sample from the central part of the pillars of the diaphragm (diaphragma pars lumbalis) which is the predilection site of Trichinella and the 1^{st} choice sampling muscle in the EU legislation. The sampling from the body of the pig should be done with a special “ice-scoop”-like forceps ensuring sampling of 1 g (alternatively 5 g in USA). For meat from horse or game the amount of tissue is 5 g. The precision of this sampling should be 1-1.15 g (EU legislation).

2. Tissue-Grinding or Blending Compartment

This compartment is thought to function as a container for the initial sampling at the slaughter line (and assignment of a bar code) and later as a meat grinder or meat blender. Following sampling, the samples are pooled to an amount of 100-500 g meat, and the pool of meat is washed in water in a grinder or blender to reduce the amount of blood (clarity of the final digest) and to lubricate the meat for the subsequent grinding or blending process. The compartment may be made in high tension steel to allow up to 500×1 g meat samples to be pressed through 3 mm holes (like a garlic press). The EU “magnetic stirrer method” requires a classic meat grinder with 2-4 mm openings or a blender. Grinding or blending of meat will increase the surface for enzymatic activity substantially and thus eliminate the need for further maceration of the samples. After pressing or blending of the meat samples, the compartment may be cleaned automatically and recycled.

3. Digestion Compartment

The meat from the grinding or blending compartment may be pressed or processed directly into a glass beaker. The digestion procedure is equivalent to the EU “Magnetic stirrer method”. For pools of 100×1 g samples a digestion fluid is prepared in the following order: Tap water (2 L, 45° C.), HCl (0.016 L, 25%), 10 g pepsin (1:10.000 National Formula), to avoid destruction of the enzyme. A liquid formulation of pepsin may substitute the pepsin powder. The digestion solution (fluid+meat) is vigorously stirred (250 rpm) on a magnetic stirrer placed in an incubator maintained at 45° C. (±1° C.). The mixture is allowed to digest for 30 min. before it is passed through the filtration compartment (se below). The digestion beaker is flushed twice to the filter with water to recover any larvae on the inside surface of the beaker.

As present, none of the methods described in the EU legislation digest more than 100 g samples, as manual handling of more than the equivalent 2 L digestion fluid is not feasible. Following robot handling of the digestion fluids this is no longer a constraint, and since sensitivity is still the same (based on 1 g samples), it opens up for larger pools e.g. 500×1 g samples and equivalent 10 L digestion fluid.

The digestion compartment may comprise a number of digestion containers. By having several digestion containers, several digestion processes can be processing at the same time. It is preferred that a conveyer, robot or another similar system is arranged so as to move a digestion container from process to process.

4. Filtration Compartment

The digestion fluid is lead through the filtration compartment comprising two nylon sieves (180 μm mesh size and 35 μm mesh size). As in the EU “magnetic stirrer technique”, a 180 μm steel sieve retain undigested pieces of fat, tendons and fascia but will let the Trichinella larvae pass. As in the EU “on filter isolation technique”, a 35 μm steel sieve, which is arranged below a 180 μm steel sieve, will retain the Trichinella larvae. The resulting filtrated liquid phase of the digestive fluid can be immediately discharged without any bio-hazards. The nylon filters are held on each side by cylinders through which the digestive fluid is passing.

5. Identification Compartment

The 35 μm nylon sieve is scanned by a digital camera under exposure with different illumination and the resulting image is computer analysed. Filters or filter parts with suspected findings are placed in labelled containers with ethanol to allow for further larval identification by a veterinarian, back-tracing of infected carcasses, and to provide conservation of larval DNA for subsequent molecular typing by a reference centre.

Compartments 2 and 3 should be made in materials that tolerate repeated cleaning at 85° C.

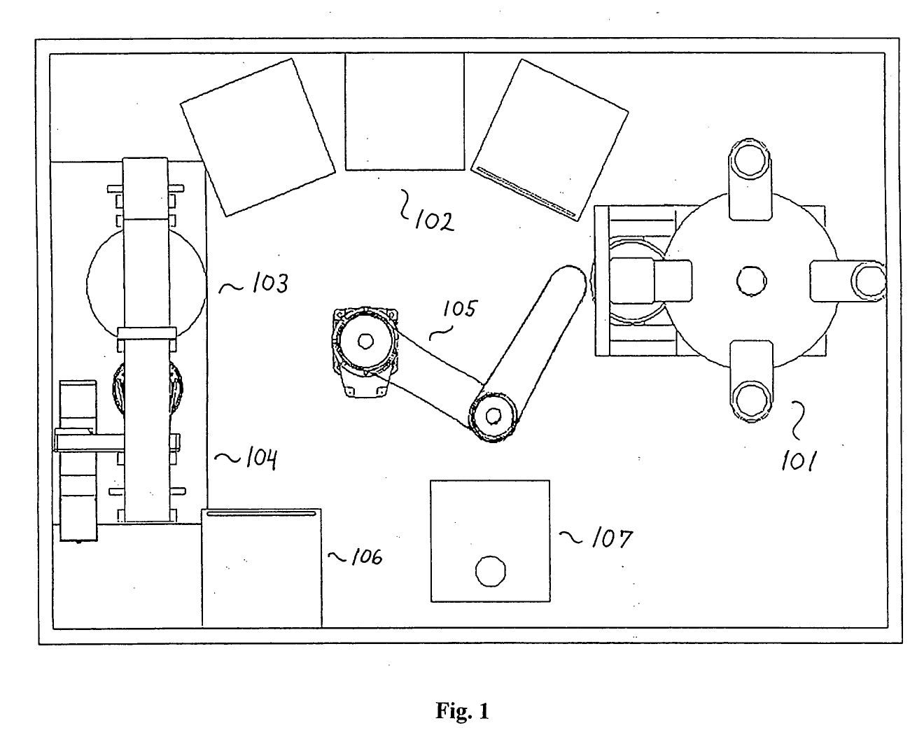

A block diagram of a detection system according to the above-discussed embodiment of the invention is illustrated in FIG. 1. The system of FIG. 1 comprises a tissue-grinding compartment 101, a digestion compartment 102, a filtration compartment 103, and a identification compartment 104. The system of FIG. 1 may further comprise a robot with a robot arm 105 or a similar device for the movement of digestion containers, an incubator 106 to be used during the digestion process, and a cleaning compartment 107 for cleaning of a digestion container before the container is used for a new process.

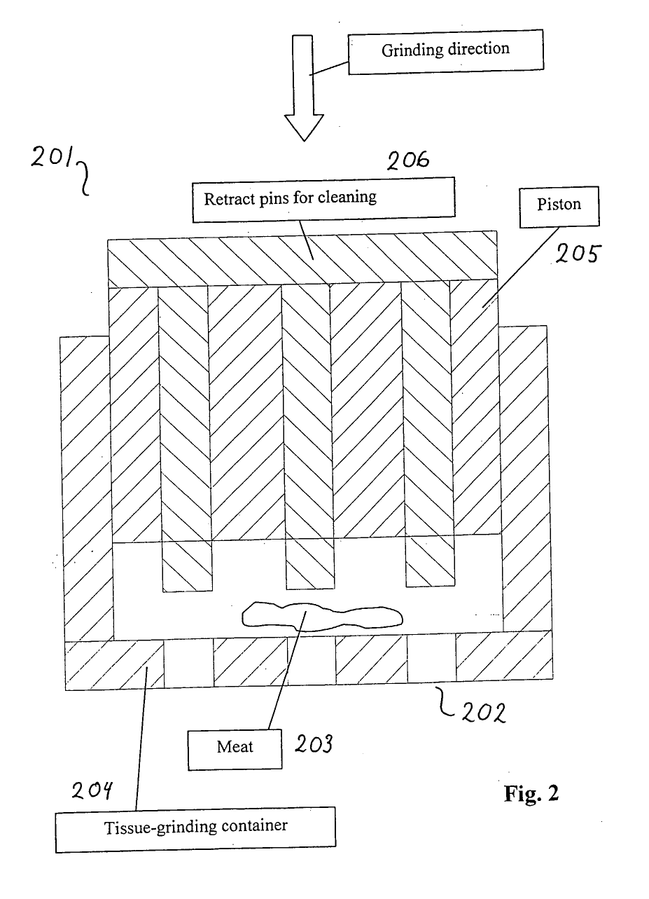

In FIG. 2 is shown a schematic view of a meat-grinding device 201 having holes 202 with a diameter of 3 mm, and which may be used in the tissue-grinding compartment 1. When pressing the meat samples 203 through the holes 202, the meat is grinded without being cut to thereby increase meat surface for the enzymatic digestion. The meat-grinding 201 device of FIG. 2 comprises a tissue-grinding container 204, a piston 205, and retract pins 206. After the grinding process, the retract pins 206 may be retracted so as to be in plane with the piston 205 to thereby obtain a plane being well suited for cleaning.

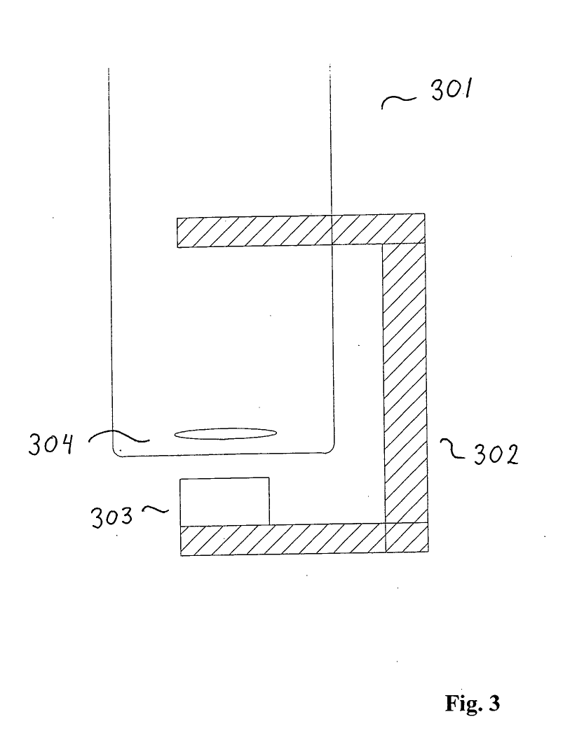

FIG. 3 shows a schematic view of an embodiment of a digestion container 301 to be used in the system of FIG. 1. The container 301 is dimensioned for pools of 100 samples of 1 g and may have a volume of 3 L. However, the digestion compartment may also be dimensioned for larger pools of meat samples, such as 500 samples of 1 g, which corresponds to 10 L of digestion fluid and a digestion container having a volume of about 15 L. The container 301 is arranged in a transportation gripper 302, whereby the container can be moved by the robot arm 105 in FIG. 1. In order to stir the digestion solution, a magnetic stirrer is provided, said magnetic stirrer comprising a permanent magnet 303 being arranged at the gripper 302 and an agitator 304 arranged at the bottom of the container 301.

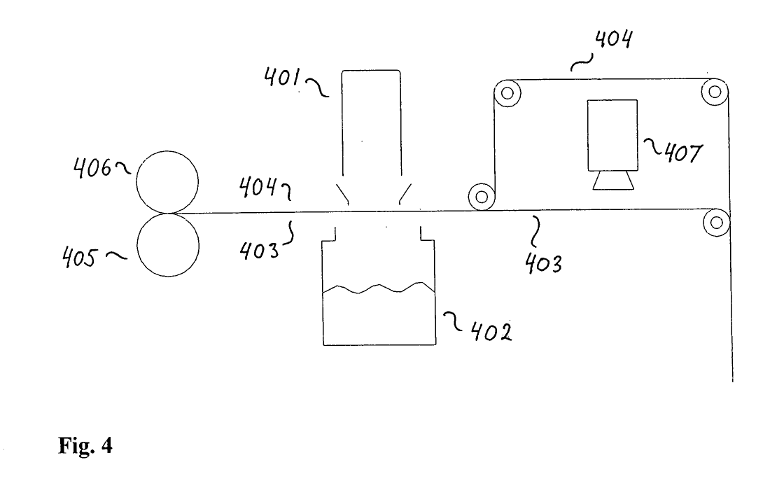

FIG. 4 shows a schematic view of an arrangement of filters, filtration arrangement and camera, which arrangement may be used in the system of FIG. 1. Here, a digestion container 401 is arranged above a waste container 402, with a first filter 403 and a second filter 404 being arrange between the digestion container 401 and the waste container 402. The second filter 404 has a mesh size of 180 μm whereby the parasite larvae to be identified can pass through the second filter, while the first filter 403 has a mesh size of 35 μm to thereby retain the parasite larvae. The filters 403, 404 are arranged on rolls 405, 406, whereby during a filtration process of digested fluid, the filters 403 and 404 automatically can be moved in the horizontal direction from the filtration arrangement to a identification compartment or area of optical inspection including a camera 407. The second filter 404 is removed from the area of optical inspection, while the first filter is arranged in front of the lens of the camera 407, whereby the images of the filtration area or areas may be recorded.

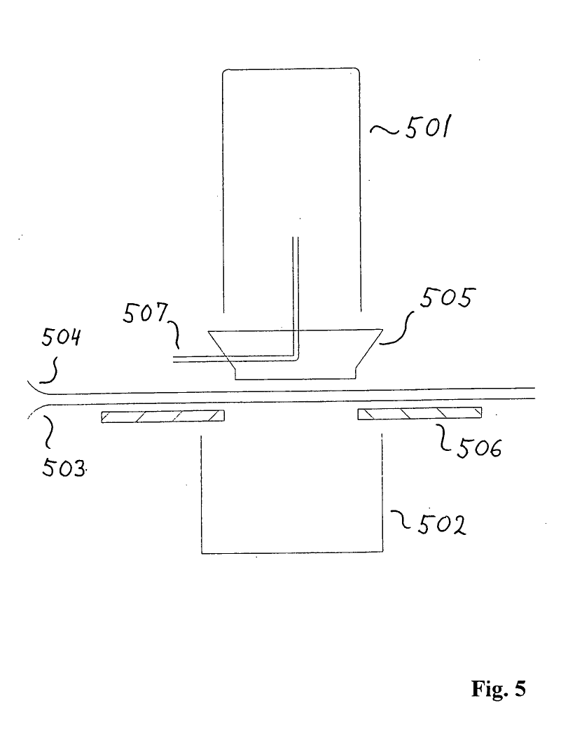

FIG. 5 is a schematic view showing a filtration arrangement, which can be used as the filtration arrangement of FIG. 4. In FIG. 5 a digestion container 501 is arranged in a funnel 505 so that the digestion solution can run from the container 501 via the funnel. 505, through the second filter 504, through the first filter 503 and into a waste container 502. A cylinder 506 is provided in order to hold the first filters 503 above the waste container 502. It is preferred that the second filter 504 having the larger mesh size of 180 μm is arranged at a distance above the first filter 503 with a mesh size of 35 μm. Flushing means or a tube 507 is arranged so that the digestion container 501 can be flushed twice with water during the filtration process in order to recover any larvae on the inside of the digestion container 501. When starting the filtration process, the digestion fluid is poured out from the digestion container 501 into the funnel 505. To pour the digestion fluid out of the container 501, the container 501 is rotated. When the container 502 has been rotated 180 degrees, the container 501 is flushed twice by using the tube 507 in the middle of the funnel.

The first and second filters, 403, 503 and 404, 504, may be commercially available filters from producer Verseidag Techfab, Geldern-Wladeck, Germany. The products are named MONODUR and are precision fabrics for screening, classifying and defined filtering for use in the foodstuff industry and chemical industry. The MONODUR filter fabrics are available with mesh openings from 5 μm to 2000 μm and are produced from monofilament wires of polyamide, polyester, polyethylene and polypropylene.

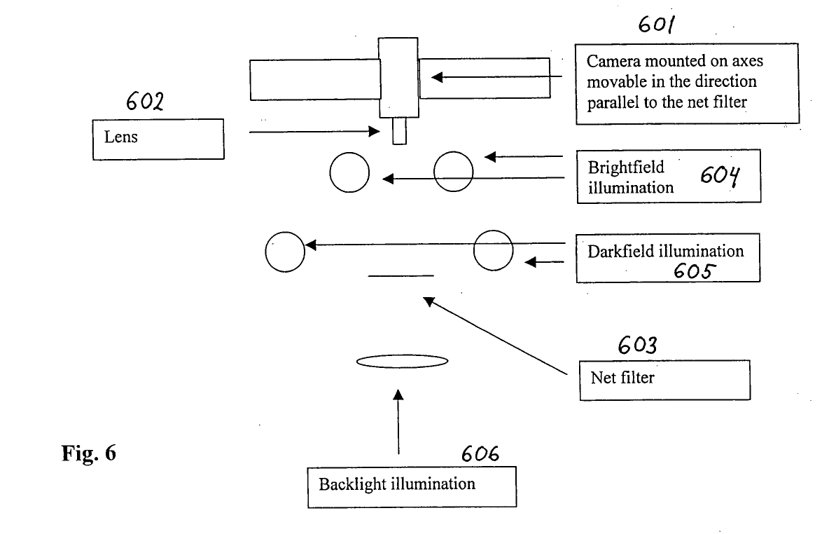

FIG. 6 shows a schematic view of a camera and illumination arrangement being part of an image acquisition system used for optical inspection of a filter according to an embodiment of the invention. The components of the image acquisition system are a camera 601 and a lens 602, which may be arranged movable in relation to the filter 603. It is preferred that the camera 601 is moved instead of moving the filter 603, since an acceleration of the filter 603 may shift the position of debris and larvae. The camera 601 may acquire a multitude of images. For each camera position a set of different illuminations may be used and for each combination of position and illumination one image may be acquired. Illumination may be e.g. bright field 604, dark field 605, diffuse, backlight 606 or combinations hereof.

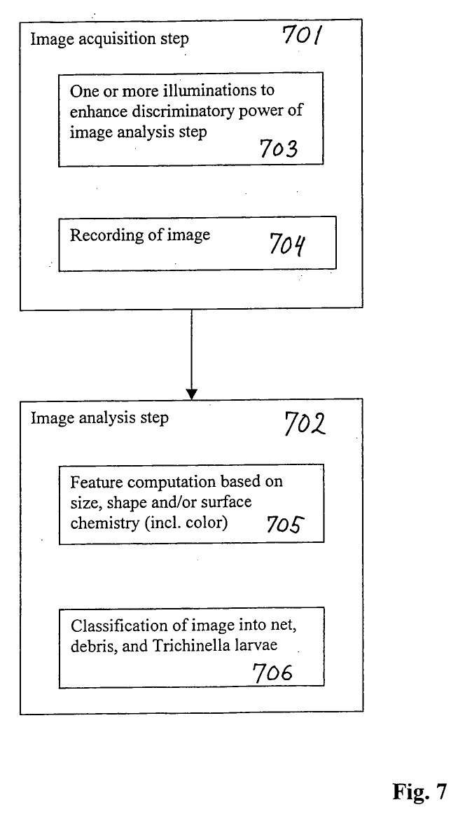

FIG. 7 is a flow chart showing steps of an identification process according to an embodiment of the invention. The identification process of FIG. 7 consists of an image acquisition step 701 followed by an image analysis step 702. In the image acquisition step, the nylon net filter or sieve with the smaller mesh size may be scanned by a camera, and the image or images may be transferred to computing equipment. The camera may be able to move in the horizontal plane (parallel to the filter or sieve being inspected) recording a multitude of images at different positions to record the relevant area of the filter with high resolution. In the image acquisition step 703, illumination may be applied on the filter or sieve to help the identification of Trichinella larvae. The illumination may be at a fixed position or it may be moved together with the camera. The illumination may be chosen to enhance the size, shape, or surface chemistry of the Trichinella larvae in contrast to the filter background and debris. For size and shape enhancement, one may choose e.g. a dark field illumination where light is applied at an angle almost parallel to the filter surface. For surface chemistry enhancement, one may choose to illuminate the sample using a diffuse illumination at one or more different wavelengths chosen from the visible, ultraviolet, and/or infrared part of the electromagnetic spectrum. One image may then be recorded 704 for each wavelength in each position providing a multi spectral image of the entire net filter. A practical set up for such a measurement can be constructed by using the apparatus and/or method disclosed in European Patent No. 1 051 660, “An apparatus and a method of recording an image of an object”, which is hereby included by reference. One illumination may be chosen to have one or more wavelengths that provide a fluorescence signal emitted from Trichinella larvae, and the camera may then be fitted with an optical filter that transmits the emitted light but absorbs or reflects the illumination wavelength(s).

The image analysis step 702 may apply standard image analysis techniques known from the literature to discriminate between net background, debris, and Trichinella larvae. Trichinella larvae and debris may be discriminated from the net background by image intensities or by textural features. Trichinella larvae may be discriminated from debris by a shape and size analysis 705 of the objects that are not net background. Shape and size features may be fed into a pattern recognition classifier 706 that will perform the actual discrimination. As a supplement or alternative to shape and size features a recorded multi spectral image may directly show differences in surface reflectance at different wavelengths for Trichinella larvae, debris and the net filter or sieve. A reflectance spectrum can be measured for each of these three classes and wavelengths with a high discriminatory power between the classes can be chosen. There are several differences in surface chemistry between the three classes one of them being the contents of carbohydrates, where Trichinella larvae has the highest contents. One may choose wavelengths where one or more will show a difference in carbohydrate contents e.g. 920 nm and 990 nm.

A preferred method is to acquire reflectance images at one or more wavelength, where the Trichinella larvae absorbs light e.g. 920 nm and 990 nm, and one or more wavelength used for normalization e.g. 870 nm. In every pixel a combination of the three reflectance values is computed e.g. by taking the ratio (reflectance at 990 nm)/(reflectance at 870 nm) or (reflectance at 920 nm)/(reflectance at 870 nm). The ratio image will then show a difference between larvae and debris/net. Object candidates assumed to be larvae are extracted by selecting pixels with ratio values that correspond to larvae. Shape parameters like contour regularity and overall shape are computed on each candidate object, and if these parameters are within a given range allowed for larvae, then the object is a detection of larvae. The shape parameters may be based on template based match or deformable models like active shape models known from the literature. Contour regularity could be based on summarizing the curvature around the contour.

Another preferred method is to acquire a multitude of images with illuminations that detect Trichinella larvae based on colour and shape e.g. using a diffuse red (630 nm) wavelength illumination, a diffuse blue (450 nm) wavelength illumination, and a darkfield illumination together. The darkfield illumination will highlight objects on the filter, and the red and blue will be used as colour features to discriminate Trichinella larvae from debris. If green illumination is chosen for the darkfield, then an ordinary RGB-camera may be used for simultaneous acquisition of the three illuminations. Otherwise the three images may be acquired in sequence.

The 20-60 μm per 20-40 μm distance between the threads in the nylon net filter or sieve (mesh size) may be used for a continuous calibration of the identification software.

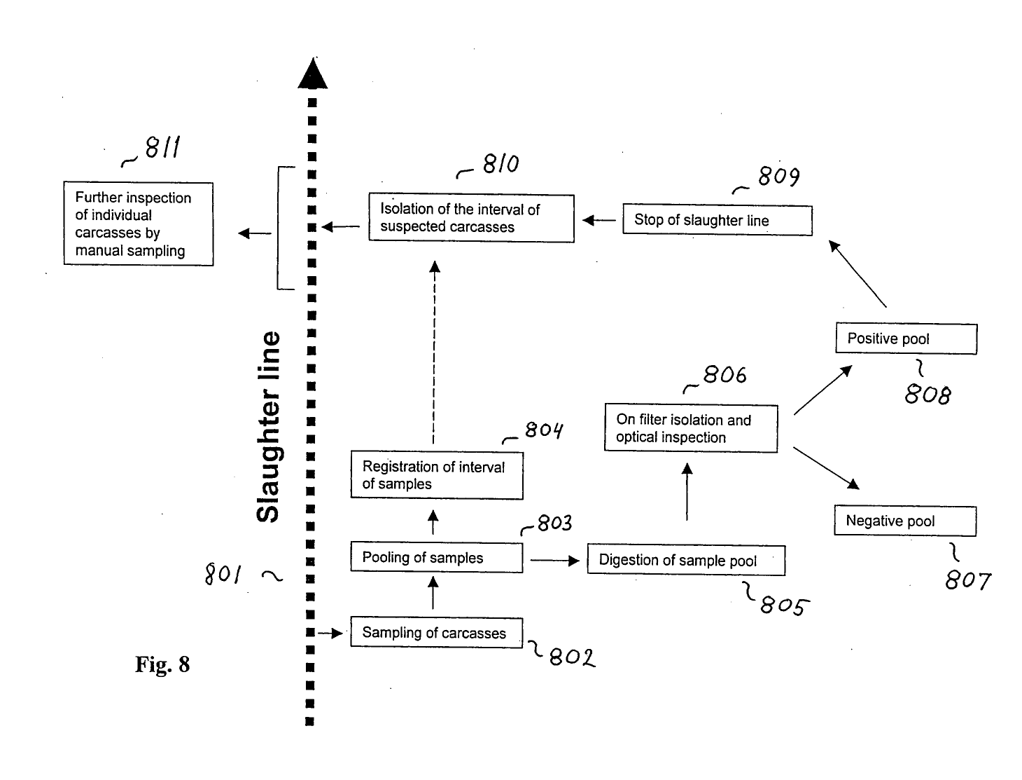

When using a detection system according to the present invention at a slaughter line, it is critical that when one or more Trichinella larvae have been detected, the carcasses from which the tested pool of meat samples originates can be traced back and isolated for examination on individual pigs. This is illustrated in the flow chart of FIG. 8.

In FIG. 8 the dotted line 801 illustrates a slaughter line where the carcasses are moved forward along the line. Here, meat samples are taken from the carcasses, step 802, and the samples are pooled together, step 803. A registration of the slaughter line interval of carcasses delivering the pooled samples is performed, step 804, and the pooled samples are digested, step 805. After the digestion, the digested fluid is filtered and the optical inspection including recording of images of the filtration area is performed, step 806. When the result of the analysis of the recorded images is that no Trichinella larvae have been detected, step 807, then no further action is taken. When the result of the analysis of the recorded images is that one or more Trichinella larvae have been detected, step 808, then the slaughter line is stopped, step 809, and an isolation of the slaughter line interval of suspected carcasses is performed, step 810. Further inspection of the individual, isolated carcasses is the performed manually, step 811.

It should be understood, that when using a Trichinella detecting system according to the present invention as illustrated in FIG. 1, it is possible to perform at proficiency test of the detection system. The proficiency test may be performed as follows:

A panel of 10 pooled samples of each 100×1 g pieces of pork tissue is used to test the proficiency of the test or detection system. Three samples among the 10 samples are added minute parts of artificially infected mice tissue containing Trichinella spiralis larvae at three different levels. The samples are sent to the respective slaughterhouses where they are subjected to digestion in the detection system. It is now the task of the detection system to identify the infected samples and the task of the service personal to preserve and quantify the number of recovered larvae. The panel including the infected samples should be prepared by an independent laboratory.

When using the principles of the identification method and system according to the present invention, the following may be taken into account:

1. Specificity (Image Analysis)

The specificity of the test may rely on the visual detection of larvae and the ability of the analysing means to distinguish Trichinella larvae from the net-background. Here, the ability of the analysing means to perform the detection may depend on the software used for analysing images recorded through the scanning process. Debris is considered a minor problem as the undigested muscle fibre fragments are much smaller than larvae. Any detection of images resembling larvae should be evaluated by a veterinarian.

2. Typing of Positive Findings

Any muscle larvae recovered by the detection system according to an embodiment of the present invention may be typed by molecular means. The grinding and digestion exposure do not influence identification by the reference molecular method (Zarlenga et al., 1999). Thus, the process allow for identification of the Trichinella larvae in any tissue, frozen or not, and thereby a risk evaluation of each positive finding.

3. Sensitivity (Mesh Size, Sample Size)

The grinding of the tissue as in compartment 1 is gentle to the Trichinella larvae. Larvae in grinded meat are virtually never damaged, whereas larvae recovered from blended tissue can suffer from the processing, especially if the tissue has been frozen. A 35 μm net will not allow even damaged larvae to pass and thereby leave the net filter without being detected. The above described detection system according to the present invention should thus be able to detect a single larva in the given pooled of meat samples. The theoretical sensitivity would then be 1 larva per gram (lpg) if one gram samples is used (EU). Under circumstances where five gram (USA) samples are preferred, e.g. in high endemic areas, the theoretical sensitivity should be 0.2 lpg.

4. Cadence

The capacity limiting process is the digestion of tissue. With a digestion time of 30 min. the limiting factor is the volume of digestion fluid that can be handled. Following present EU legislation where the maximal pool size is 100 samples (100 g meat+2 L fluid) the capacity will be 200 samples per hour per digestion container. It may be possible to combine several digestion containers. To allow for future changes in the EU legislation, both the tissue grinding compartment and the digestion compartment should have capacity to handle up to 500 samples (500 g tissue) and 10 L digestive fluid.

5. Optimal Capacity and Flexibility

If the detection system of the invention is constructed with several digestion compartments the capacity may be higher than 1000 samples per hour, but due to the fast speed at the slaughter line in meat processing plants, the meat industry advice that 500 samples would be the optimal number. This might be highly variable in between slaughterhouses and collaboration with the meat industry in EU and US may determine the range of optimal capacity. Compartmentalization will provide flexibility to the detection system.

6. Laboratory Implementation

Since the detection system of the invention may be fully automated, technical personal may only be needed when the digestion is initiated, for refilling of pepsin and HCl, and veterinary staff in case of positive findings.

7. Process Implementation and Animal ID

After collection of samples at the slaughter line the individual pool of samples may be assigned a bar code that may be read by the detection system. In this way, each reaction can easily be traced back to the interval of pigs in the slaughter line that it represents. The increasing use of animal identification systems will facilitate tracing back in case of positive findings.

8. Customisation for Various Epidemiological Scenarios

In areas where trichinellosis is highly endemic, larger but fewer samples are often prescribed in the legislation. Thus, in USA 20×5 g is used whereas 100×1 g is used in EU. For the detection system of the invention, samples of respectively 500×1 g or 100×5 g may be relevant.

While the invention has been particularly shown and described with reference to particular embodiments, it will be understood by those skilled in the art that various changes in form and details may be made therein without departing from the spirit and scope of the invention, and it is intended that such changes come within the scope of the following claims.

1.-62. (canceled)

63. A method for detection of Trichinella larvae in meat samples comprising:
a) digesting an amount of meat to be tested for Trichinella larvae, said digestion process resulting in a digested fluid,
b) filtering the digested fluid by use of a first filter being designed to retain Trichinella larvae, and
c) analysing by use of optical inspection the area of the first filter being used for the filtration of the digested fluid, said analysing being directed against identification of Trichinella larvae, wherein
the optical inspection comprises recording several images of the filtration area of the first filter being used for the filtration process, with a multitude of the recorded images representing a set of different illuminations of the filtration area of the first filter, and wherein the set of illuminations used for the filtration area comprises diffuse illumination and/or a dark field illumination.

64. A method according to claim 63, wherein the set of illuminations used for the filtration area comprises diffuse illumination at several different wavelengths.

65. A method according to claim 63, wherein the set of illuminations used for the filtration area further comprises a bright field illumination.

66. A method according to claim 63, wherein for the dark field illumination light is applied almost parallel to the filter surface.

67. A method according to claim 63, wherein the wavelength(s) of the illumination or diffuse illumination is/are selected from a list of wavelengths representing the visible, the ultraviolet and the infrared part of the electromagnetic spectrum.

68. A method according to claims 63, wherein the set of illuminations used for the filtration area comprise one illumination or diffuse illumination being selected to have a wavelength that provides a fluorescence signal emitted from Trichinella larvae.

69. A method according to claim 68, wherein the wavelength of the illumination to provide the fluorescence signal is selected in the ultraviolet part of the electromagnetic spectrum.

70. A method according to claim 63, wherein the set of illuminations used for the filtration area comprises illumination with one or more wavelengths being selected to show a difference in carbohydrate contents of Trichinella larvae, debris and the filter.

71. A method according to claim 70, wherein the selected wavelength(s) include(s) the wavelength(s) 920 nm and/or 990 nm.

72. A method according to claim 63, wherein reflectance images are acquired at one or more wavelengths where the Trichinella larvae absorbs light, and at one or more wavelengths used for normalization of the wavelengths for which the Trichinella larvae absorbs light.

73. A method according to claim 72, wherein the wavelength(s), for which the Trichinella larvae absorbs light, include(s) the wavelength(s) 920 nm and/or 990 nm, and the wavelength(s) used for normalization include(s) the wavelength 870 nm.

74. A method according to claim 63, wherein the set of illuminations used for the filtration area comprises a diffuse red (630 nm) wavelength illumination, a diffuse blue (450 nm) wavelength illumination, together with a dark field illumination.

75. A method according to claim 74, wherein green illumination is chosen for the dark field illumination.

76. A system for detection of Trichinella larvae in meat, said system comprising:
a digestion container for digesting an amount of meat to be tested for Trichinella larvae, said digestion process resulting in a digested fluid,
a first filter for filtering the digested fluid, said first filter being designed to retain Trichinella larvae,
an analyser for analysing by use of optical inspection the area of the first filter being used for the filtration of the digested fluid, said analysing being directed against identification of Trichinella larvae, and
one or more illumination devices for providing different illuminations of the filtration area of the first filter, wherein
the analyser comprises an image recorder for recording one or more images of the filtration area of the first filter being used for said filtration process, with a multitude of the recorded images representing a set of different illuminations of the filtration area of the first filter, and wherein the set of illuminations used for the filtration area comprises diffuse illumination and/or a dark field illumination.

77. A system according to claim 76, wherein the one or more illumination devices for providing different illuminations of the filtration area is adapted for providing illumination or diffuse illumination at several different wavelengths.

78. A system according to claim 76, wherein the set of illuminations used for the filtration area further comprises a bright field illumination.

79. A system according to claim 76, wherein for the dark field illumination light is applied almost parallel to the filter surface.

80. A system according to claim 76, wherein the wavelength(s) of the illumination or diffuse illumination is/are selected from a list of wavelengths representing the visible, the ultraviolet and the infrared part of the electromagnetic spectrum.

81. A system according to claim 76, wherein the set of illuminations used for the filtration area comprise one illumination or diffuse illumination being selected to have a wavelength that provides a fluorescence signal emitted from Trichinella larvae.

82. A system according to claim 81, wherein the wavelength of the illumination to provide the fluorescence signal is selected in the ultraviolet part of the electromagnetic spectrum.

83. A system according to claim 76, wherein the set of illuminations used for the filtration area comprises illumination with one or more wavelengths being selected to show a difference in carbohydrate contents of Trichinella larvae, debris and the filter.

84. A system according to claim 83, wherein the selected wavelength(s) include(s) the wavelength(s) 920 nm and/or 990 nm.

85. A system according to claim 76, wherein reflectance images are acquired at one or more wavelengths where the Trichinella larvae absorbs light, and at one or more wavelengths used for normalization of the wavelengths for which the Trichinella larvae absorbs light.

86. A system according to claim 85, wherein the wavelength(s), for which the Trichinella larvae absorbs light, include(s) the wavelength(s) 920 nm and/or 990 nm, and the wavelength(s) used for normalization include(s) the wavelength 870 nm.

87. A system according to claim 76, wherein the set of illuminations used for the filtration area comprises a diffuse red (630 nm) wavelength illumination, a diffuse blue (450 nm) wavelength illumination, together with a dark field illumination.

88. A system according to claim 87, wherein green illumination is chosen for the dark field illumination.
